(19) United States
(12) Patent Application Publication
(10) Pub. No.: US 2026/0130579 A1
(43) Pub. Date: May 14, 2026

(54) CONVERTER DEVICE FOR SIDE VIEWING WITH AN ENDOSCOPE

(71) Applicant: Loma Linda University, Lomda Linda, CA (US)

(72) Inventor: Gautam Nagendra, Loma Linda, CA (US)

(21) Appl. No.: 19/480,724

(22) PCT Filed: Apr. 26, 2024
(86) PCT No.: PCT/US2024/026580

§ 371 Date: Oct. 31, 2025

Related U.S. Application Data
(60) Provisional application No. 63/499,196, filed on Apr. 28, 2023.

(51) Int. Cl. A61B 1/00

(52) U.S. Cl. CPC A61B 1/00183; A61B 1/00096; A61B 1/00101; A61B 1/00105

(57) ABSTRACT

Disclosed herein is an endoscope converter, which can be used with an endoscope. The endoscope converter includes an attachment portion, a support member, and a mirror member. The attachment portion is operable to removably couple the support member to the endoscope. The mirror member has a first end, a second end, and an outer surface. The first end of the mirror is operably coupled to the support member such that the mirror member can rotate about the first end of the mirror member. The outer surface of the mirror member includes a mirror.

### FIELD

The present disclosure relates to endoscopes for use in medical procedures. In particular, the present disclosure relates to an endoscope converter device that temporarily allows side viewing while using an endoscope.

### BACKGROUND

Duodenoscopes are specialized endoscopes that gastroenterologists use to perform endoscopic retrograde cholangiopancreatography (ERCP) procedures. Endoscopes are devices that contain a forward viewing camera at the end of a flexible tube and are used in multiple fields for minimally invasive procedures. Endoscopes contain a forward-facing camera, which provides the proceduralist with direct visualization during forward movement as the endoscope is advanced into the patient. Additionally, instruments (e.g., tools) introduced into the instrument channel of the endoscope exit the distal end of the endoscope parallel to the view of the camera, which is forward facing. Duodenoscopes, on the other hand, contain a side viewing camera, which is ideal for ERCP procedures that may include manipulation of the biliary tree for conditions such as gallstones stuck in the duct that need to be removed. Additionally, instruments introduced into the instrument channel exit the distal end of the duodenoscope perpendicular to the view of the camera. In some cases, an elevator can be adjusted to provide variability in the instrument exit angle. However, when using a duodenoscope, the proceduralist does not have direct visualization of forward movement when inserting or otherwise advancing the duodenoscope. Instead, the proceduralist must rely on the side view from the side viewing camera and the tactile feel of resistance. Although proceduralists are extremely skilled, the lack of forward visualization while manipulating the scope during ERCP increases the chance for scope related damage or perforation injury to the patient. On the other hand, when using an endoscope, the proceduralist has direct visualization of forward movement when inserting or otherwise advancing the endoscope; however, the proceduralist does not have side visualization.

With a rising incidence of obesity in the pediatrics, there has been a greater incidence of biliary disease (such as gallstones) in the adolescent subpopulation (Walker S K, Maki A C, Cannon R M, et al., Surgery. 2013; 154 (4): 927-933. doi: 10.1016/j.surg.2013.04.040.) Similarly, there has been a rise in ERCP utilization in the pediatric population as well (Pant C, Sferra T J, Barth B A, et al. Trends in endoscopic retrograde cholangiopancreatography in children within the United States, 2000-2009. J Pediatr Gastroenterol Nutr. 2014; 59(1):57-60. doi: 10.1097/MPG.0000000000000333). Given children generally have smaller and more fragile organ systems, it is optimal to mitigate procedural risk as much as possible. The temporary conversion of a forward viewing endoscope to provide a side-facing view would be useful in the pediatric population where the gastrointestinal tract may be at higher risk for endoscope and manipulation related trauma.

### SUMMARY

Some aspects of the present disclosure provide an endoscope converter and methods of use thereof. One aspect of the present disclosure includes an endoscope converter for use with an endoscope. The endoscope converter includes an attachment portion, a support member, and a mirror member. The attachment portion can removably couple the support member to the endoscope. The mirror member includes a first end, a second end, and an outer surface. The first end of the mirror member is operably coupled to the support member so that the mirror is operable to rotate about the first end. The outer surface of the mirror member includes a mirror.

In some aspects, the mirror member is aligned with an optical lens of a camera of the endoscope when the endoscope converter is removably coupled to a distal end of the endoscope. For example, a longitudinal axis of the mirror member can be coplanar with a longitudinal axis of the optical lens of the camera. In some aspects, the mirror member is operable to rotate between a lowered position and a raised position. For example, when the mirror member is in the lowered position, the mirror member can allow a forward view through an optical lens of a camera of the endoscope. When the mirror member is in the raised position the mirror member can reflect at least a portion of the forward view to provide a side view. In some aspects, the angle between a longitudinal axis of the mirror member and a longitudinal axis of the support member defines an angle of rotation. For example, rotating the mirror member changes the angle of rotation. In some instances, the angle of rotation of the mirror member can be up to 52.5-degrees.

In some embodiments, the endoscope converter includes an elevator member that has a first end, a second end, and an outer surface. The first end of the elevator member is operably coupled to the support member so that the elevator member can rotate about the first end. The outer surface of the elevator member is operable to position an instrument. In some aspects, when the endoscope converter is removably coupled to a distal end of the endoscope, the elevator member is aligned with an instrument channel of the endoscope. In some aspects, a longitudinal axis of the elevator member is coplanar with a longitudinal axis of the instrument channel. In some aspects, the angle between a longitudinal axis of the elevator member and a longitudinal axis of the support member defines an angle of elevation. For example, rotating the elevator member changes the angle of elevation.

In some embodiments, the endoscope converter includes a first tension member and a second tension member. The first tension member has a proximal end and a distal end. The distal end of the first tension member is in communication with the mirror member, whereby actuating the first tension member causes the mirror member to rotate. For example, the mirror member can rotate between a lowered position and a raised position. The second tension member has a proximal end and a distal end. The distal end of the second tension member is in communication with the elevator member, whereby actuating the second tension member causes the elevator member to rotate. For example, the elevator member can rotate between a lowered position and a raised position. In some embodiments, the endoscope converter includes a first lever and a second lever. The first lever can be connected to the proximal end of the first tension member, whereby manipulating the first lever causes the first tension member to actuate. The second lever can be connected to the proximal end of the second tension member, whereby manipulating the second member causes the second tension member to actuate.

In some embodiments, the attachment portion includes a first semi-cylindrical surface and a second semi-cylindrical surface. When the endoscope converter is removably coupled to the distal end of the endoscope, the first semi-cylindrical surface and the second semi-cylindrical surface are positioned on opposite sides of an outer cylindrical surface of the endoscope.

In some embodiments, the endoscope converter includes a light source. The light source defines a longitudinal axis and is configured to illuminate an area aligned with the longitudinal axis. In some embodiments, when the attachment portion of the endoscope converter is removably coupled to the endoscope and the light source is illuminated, the longitudinal axis of the light source is generally perpendicular to a longitudinal axis of the distal end of the endoscope.

In some embodiments, the support member defines a planar surface. In some aspects, when the endoscope converter is removably coupled to the distal end of the endoscope, the planar surface of the support member is generally parallel to the longitudinal axis of the distal end of the endoscope. In some aspects, when the endoscope converter is removably coupled to the distal end of the endoscope, the support member extends laterally outward from the distal end of the endoscope. In some aspects, the support member includes a passageway such that the support member is cleft.

Also disclosed is a method of using the endoscope converter. One aspect of the present disclosure includes using the endoscope converter to provide side viewing with the endoscope. The endoscope converter can include an attachment portion, a support member, and a mirror member. The attachment portion can removably couple the support member to the endoscope. The mirror member can include a first end, a second end, and an outer surface. The first end of the mirror member can be operably coupled to the support member so that the mirror is operable to rotate about the first end. The outer surface of the mirror member can include a mirror.

The method includes attaching the endoscope converter to the distal end of the endoscope that has an optical lens associated with a camera. Additionally, the method includes inserting the distal end of the endoscope into a patient and advancing the endoscope to a desired location with the patient. The method includes rotating the mirror member. The method can also include removing the endoscope from the patient.

In some aspects, when advancing the endoscope to the desired location within the patient, the mirror member is in a lowered position to allow a forward view through the optical lens of the endoscope. In some aspects, after advancing the endoscope to the desired location within the patient, the mirror member can be rotated into a raised position to at least partially reflect the forward view through the optical lens to provide a side view.

In some aspects, the method includes actuating an elevator member that has a first end, a second end, and an outer surface. The first end of the elevator member can be operably coupled to the support member such that the elevator member is operable to rotate about the first end of the elevator member. The outer surface of the elevator member can be operable to position an instrument.

In some aspects, the method includes inserting the distal end of the endoscope into the mouth of the patient. The method can also include advancing the distal end of the endoscope through the mouth of the patient, through the throat of the patient, through the stomach of the patient, and into the duodenum of the patient.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1A is a perspective view of an exemplary embodiment of an endoscope converter connected to an endoscope, according to an exemplary embodiment.

FIG. 1B is a front view of the exemplary embodiment of the endoscope converter connected to the distal end of the endoscope.

FIG. 1C is a top view of the exemplary embodiment of the endoscope converter connected to the distal end of the endoscope.

FIG. 1D is a side view of the exemplary embodiment of the endoscope converter with the mirror in a lowered position.

FIG. 1E is a side view of the exemplary embodiment of the endoscope converter with the mirror in a raised position.

FIG. 2A is a perspective view of an exemplary embodiment of an endoscope converter, coupled to the distal end of the endoscope, with the mirror in a lowered position and the elevator in a lowered position.

FIG. 2B is a perspective view after the mirror has been transitioned into a raised position and the elevator has been transitioned into a raised position.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

Provided herein is an endoscope converter device and methods of use to provide side viewing with a forward-facing endoscope. The converter device includes a mirror that can rotate between a lowered position and a raised position. In the lowered position, the mirror does not obstruct or otherwise reflect the forward-facing view through the camera of the endoscope, which allows for traditional forward viewing through the camera. In the raised position, the mirror reflects the forward-facing view of the camera, which provides a side view. In this manner, the endoscope converter device augments a forward-facing endoscope with side viewing capabilities.

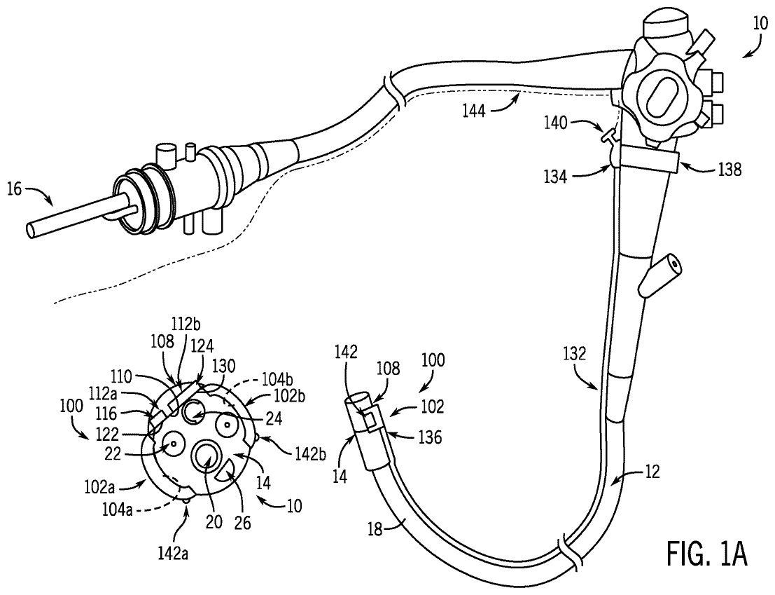

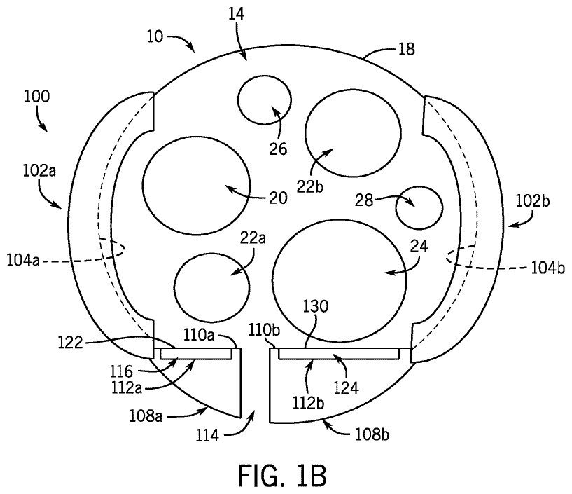

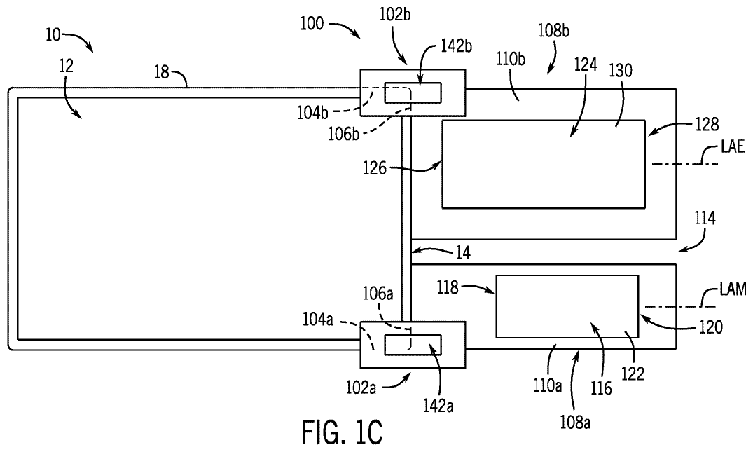

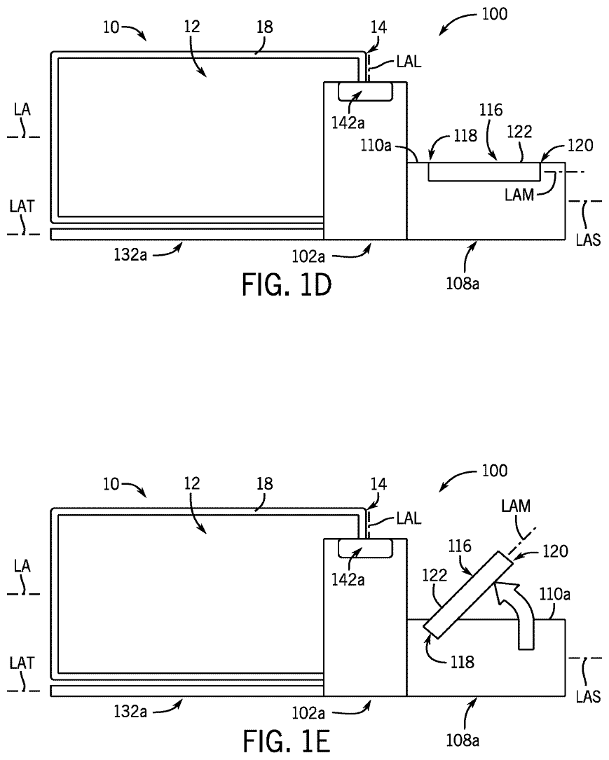

As illustrated in FIGS. 1A-1E, the converter device includes an attachment or attachment mechanism, which can removably couple the converter device to the distal end of an endoscope. A platform (e.g., lateral member) extends laterally outward from the attachment, such that the platform extends laterally outward from the distal end of the endoscope when the converter device is removably coupled to the endoscope. The platform can support both a mirror and an elevator. One or more tension rods can extend from the mirror and/or elevator to a proximal location on the endoscope, such that each tension rod can be actuated to cause the mirror and/or elevator to raise and/or lower. One or more proximal levers, which can be located at the proximal end of the one or more tension rods, can be configured to control (e.g., raise and/or lower) the mirror and/or elevator (e.g., by actuating the one or more tension rods). In some aspects, the mirror and the elevator can be independently raised and/or lowered. One or more light sources can be coupled to the attachment mechanism and configured to shine light radially outward from the endoscope. One or more proximal dials can control the light sources (e.g., on, off) and also adjust the intensity of the light sources. In some aspects, the converter device-which can include, for example, the attachment, platform, mirror, elevator, one or more tension rods and one or more proximal dials, and light sources and one or more proximal dials-can be replaceable. For example, a first converter device (e.g., a used device) can be removed from the endoscope and a second converter device (e.g., a new or unused device) can be removably coupled to the endoscope. In some aspects, the converter device can be disposable (e.g., the used device can be discarded).

One end of the mirror is connected (e.g., hinged) to the platform, such that the mirror can rotate about its connection to the platform. In this manner, the mirror can rotate between a lowered position (as illustrated for example in FIG. 1D) and a raised position (as illustrated for example in FIG. 1E). The mirror is aligned with the camera of the endoscope, such that when the mirror is in a raised position the mirror provides a side view (e.g., radially outward from the longitudinal axis of the distal end of the endoscope) by reflecting at least a portion of the forward-facing view from the camera. When the mirror is in a lowered position, the mirror allows a forward-facing view through the camera because the mirror does not reflect the view from the camera when the mirror is in the lowered position.

One end of an elevator is connected (e.g., hinged) to the platform, such that the elevator can rotate about its connection to the platform. In this manner, the elevator can rotate between a lowered position and a raised position. The elevator is aligned with the instrument channel of the endoscope, such that when the elevator is in a raised position, the elevator can position a tool extending through the outlet of the instrument channel at an angle with respect to the longitudinal axis of the instrument channel. When the elevator is in a lowered position, the elevator positions a tool extending through the outlet of the instrument channel at no angle.

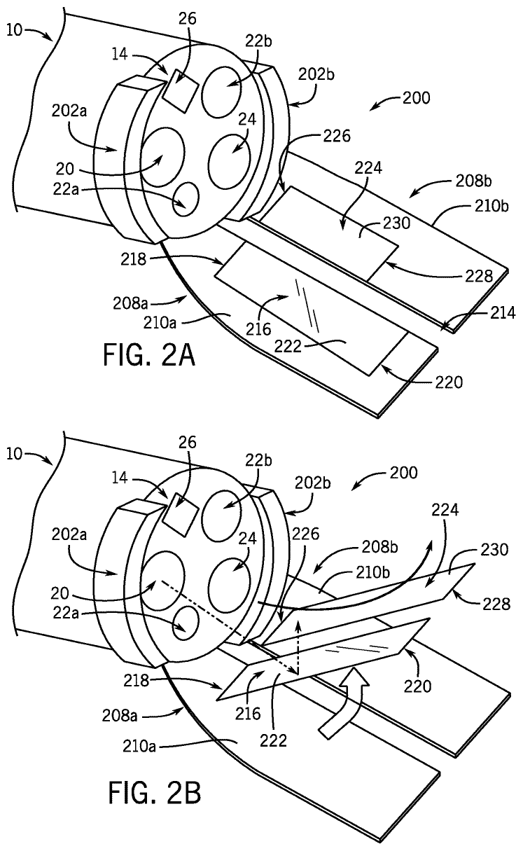

As illustrated in FIGS. 2A-2B, the mirror can be in lowered position (as illustrated for example in FIG. 2A) to allow for a forward-facing view from the camera of the endoscope or in a raised position (as illustrated for example in FIG. 2B) to reflect the forward-facing view to provide a side view. Independent of the position of the mirror, the elevator can be in a lowered position (as illustrated for example in FIG. 2A) to allow for tools to advance straightforward from the outlet of the instrument channel or in a raised position (as illustrated for example in FIG. 2B) to position a tool extending outward from the outlet of the instrument channel at an angle.

The endoscope converter device described herein may provide significant benefits over conventional endoscopes and/or conventional duodenoscopes. Because the endoscope converter device disclosed herein can transition between allowing a forward view and providing a side view, it provides significant advantages over both conventional duodenoscopes and conventional endoscopes.

As one example of benefits over conventional devices, the endoscope converter device may reduce the risk for duodenoscope related trauma, such as perforation. Conventional duodenoscopes do not allow for direct visualization of forward movement when advancing or manipulating the scope. Instead, the endoscopist must rely on the side view and the tactile feel of resistance as the duodenoscope traverses the lumen or abuts the luminal walls. The lack of forward visualization increases the chance for duodenoscope related trauma, procedural time, and perforation injury. However, the endoscope converter device disclosed herein allows for direct visualization of forward movement. For example, the mirror can be placed in the lowered position to allow forward viewing through the endoscope when advancing the endoscope in the body of the patient. This forward visualization can reduce the risk for trauma, such as perforation, which may occur more frequently with a conventional duodenoscope.

Conventional endoscopes do not allow for side viewing, which can be required while performing certain techniques or procedures such as, for example, ERCP. The endoscope converter device disclosed herein allows for side visualization after the endoscope has reached a desired location in the body of the patient. For example, the mirror can be placed in the raised position to reflect the forward view of the endoscope to provide a side view.

As another example of benefits over conventional devices, the endoscope converter device may reduce procedure time. For example, forward viewing can be beneficial when advancing the endoscope through the body of the patient while side viewing can be beneficial in certain procedures, such as ERCP. The ability to transition between a forward view and a side view may reduce procedure time, such as by reducing the time required to advance the distal end of the duodenoscope to a desired location within the patient to perform ERCP.

As another example of benefits over conventional devices, the endoscope converter device may reduce the risk of duodenoscope contamination. For example, conventional duodenoscopes may retain microbes in difficult to clean locations such as, for example, the elevator mechanism. In some aspects, the endoscope converter device disclosed herein can be removable, replaceable, and/or disposable, which may reduce the risk of contamination. Moreover, the endoscope converter device may reduce resources required to decontaminate a conventional duodenoscope. For example, studies estimate that even partially disposable duodenoscopes require significant resources (e.g., approximately 92 liters of water) for decontamination. The disposable nature of the endoscope converter device disclosed herein may reduce or eliminate the resources required for decontamination.

FIGS. 1A-1E illustrate an endoscope converter 100 (e.g., endoscope viewing converter) according to an exemplary embodiment. The endoscope converter 100 can be removably coupled to an endoscope 10, as illustrated for example in FIGS. 1A-1E, such that the endoscope converter 100 can be used in conjunction with the endoscope 10 as described herein. Additionally, the endoscope converter 100 can be decoupled (e.g., removed) from the endoscope 10. When removably coupled to an endoscope 10, the endoscope converter 100 can provide alternative viewing (e.g., side viewing) from the endoscope 10. For example, although an endoscope 10 is forward-facing and provides forward viewing, the endoscope converter 100 can facilitate side viewing with the endoscope 10. For example, the side view can be radially-outward from an axis of the typical forward facing view (e.g., the longitudinal axis LA of the endoscope 10 at its distal end 14).

The endoscope 10 is an elongate medical device that includes an insertion tube 12 that defines a longitudinal axis LA and has a proximal end 16, a distal end 14, and an outer cylindrical surface 18, as illustrated for example in FIG. 1A. The longitudinal axis LA refers to an axis along the centerline of the endoscope 10, between the proximal end 16 and the distal end 14. The longitudinal axis LA follows the path of the endoscope 10, which may be bent, curved, or otherwise manipulated. Therefore, when the endoscope 10 is manipulated during use, the longitudinal axis LA varies with the centerline of the endoscope 10. The longitudinal axis LA of the endoscope 10 is illustrated, for example in FIGS. 1D-1E, which illustrate an endoscope converter 100 removably coupled to a distal end 14 of an endoscope 10.

In some examples, as illustrated in FIGS. 1A-1B, the distal end 14 of the endoscope 10 can include an objective lens 20, one or more light-guide lenses 22 (e.g., 22a, 22b), an instrument channel outlet 24 (e.g., biopsy channel), an air/water nozzle 26, and/or an auxiliary water channel outlet 28. In some aspects, the objective lens 20 is associated with a camera that provides viewing from the distal end 14 of the endoscope 10. For example, the camera can provide viewing through the objective lens 20. In some aspects, the one or more light-guide lenses 22 are associated with one or more corresponding light sources to provide lighting from the distal end 14 of the endoscope 10. For example, light from the one or more light sources can be projected through the one or more corresponding light-guide lenses 22. In some aspects, the instrument channel outlet 24 is associated with an instrument channel (e.g., a lumen) that can receive instruments therethrough. For example, an instrument can be inserted into a proximal end of the instrument channel, advanced through the instrument channel, and exit the instrument channel at the instrument channel outlet 24. After using the instrument, the instrument can be retracted through the instrument channel outlet 24 and into the instrument channel, retracted through the instrument channel to the proximal end of the instrument channel, and removed from the instrument channel. In some aspects, the air/water nozzle 26 is associated with an air and/or water supply. For example, air and/or water (e.g., supplied from the corresponding air and/or water supply) can be discharged though the air/water nozzle 26. In some aspects, the auxiliary water channel outlet 28 is associated with an auxiliary water channel that can direct water therethrough. For example, water supplied into the auxiliary water channel can exit through the auxiliary water channel outlet 28.

The endoscope converter 100 can include an attachment portion 102, a support member 108, a mirror member 116, an elevator member 124, and/or a light source 142. For example, the attachment portion 102 can removably couple the endoscope converter 100 to the endoscope 10. The support member 108 can extend laterally outward from the attachment portion 102. The support member 108 can support a mirror member 116, such that the mirror member 116 can rotate. Similarly, the support member 108 can support an elevator member 124 such that the elevator member 124 can rotate. The attachment portion 102 can include one or more light sources 142, which can illuminate at least a portion of an area that is visible when the mirror member 116 is rotated to provide side viewing. Each of these components are discussed in more detail below.

The disclosure turns now to the attachment portion 102 (e.g., 102a, 102b), which can removably couple the endoscope converter 100 to the distal end 14 of the endoscope 10. For example, the attachment portion 102 can be removably coupled to the distal end 14 of the endoscope 10, thereby removably coupling the endoscope converter 100 to the endoscope 10. The attachment portion 102 supports the support member 108, which is discussed below. Additionally, the attachment portion 102 can house or otherwise support one or more light sources 142, which is discussed below.

The attachment portion 102 can define a semi-cylindrical surface 104 (e.g., 104a, 104b), illustrated by dashed lines (which represents the internal semi-cylindrical surface 104 at the outermost diameter of the endoscope 10) on the top view in FIG. 1C. The semi-cylindrical surface 104 of the attachment portion 102 can abut, in whole or in part, the endoscope 10 (e.g., outer cylindrical surface 18) when the attachment portion 102 is removably coupled to the endoscope 10. The semi-cylindrical surface 104 of the attachment portion 102 abutting the outer cylindrical surface 18 of the endoscope 10 can inhibit the endoscope converter 100 from moving (e.g., translating, rotating) with respect to the distal end 14 of the endoscope 10.

The attachment portion 102 can define a planar surface 106 (e.g., 106a, 106a), illustrated by dashed lines (which represents the internal planar surface 106) on the top view in FIG. 1C. The planar surface 106 of the attachment portion 102 can abut, in whole or in part, the distal end 14 (e.g., planar surface containing the objective lens 20, one or more light-guide lenses 22, an instrument channel outlet 24, an air/water nozzle 26, and/or an auxiliary water channel outlet 28) of the endoscope 10 when the attachment portion 102 is removably coupled to the distal end 14 of the endoscope 10. The planar surface 106 of the attachment portion 102 abutting the distal end 14 the endoscope 10 can inhibit the endoscope converter 100 from moving (e.g., translating, rotating) with respect to the distal end 14 of the endoscope 10.

In some embodiments, the semi-cylindrical surface 104 of the endoscope converter 100 is generally perpendicular to the planar surface 106 of the endoscope converter 100. In this manner, the semi-cylindrical surface 104 can abut, in whole or in part, the outer surface (e.g., outer cylindrical surface) of the endoscope 10 and the planar surface 106 can abut, in whole or in part, the distal end 14 (e.g., planar surface) of the endoscope 10 to inhibit the endoscope converter 100 from moving (e.g., translating, rotating) with respect to the distal end 14 of the endoscope 10.

In some embodiments, the attachment portion 102 can include more than one attachment portion 102 (e.g., 102a, 102b), which each define a respective inner surface 104 (e.g., 104 a, 104 b) and/or a respective planar surface 106 (e.g., 106a, 106a), as previously described. For example, as illustrated in FIGS. 1A-1C, the attachment portion 102 can include a first attachment portion 102a and a second attachment portion 102b. The first attachment portion 102a can define an inner surface 104a and the second attachment portion 102b can define an inner surface 104b. When the attachment portion 102 is removably coupled to the endoscope 10, the first attachment portion 102a is positioned on the opposite side of the outer surface of the endoscope 10 from the second attachment portion 102b. In this manner, the semi-cylindrical surfaces 104a, 104b counteract forces applied to the endoscope converter 100 to inhibit movement (e.g., translation, rotation) of the endoscope converter 100 with respect to the distal end 14 of the endoscope 10.

The disclosure turns now to the support member 108 (e.g., 108a, 108b), which extends laterally outward from the attachment portion 102 as illustrated in FIGS. 1C-1E. For example, when the endoscope converter 100 (e.g., the attachment portion 102) is removably coupled to the distal end 14 of the endoscope 10, the support member 108 can extend laterally outward from the distal end 14 of the endoscope 10. The support member 108 can support the mirror member 116 and/or the elevator member 124, as discussed below.

The support member 108 can define a longitudinal axis LAS, as illustrated in the side views in FIGS. 1D-1E. In some aspects, when the endoscope converter 100 is removably coupled to the endoscope 10, the longitudinal axis LAS of the support member 108 can be generally parallel to the longitudinal axis LA of the distal end 14 of the endoscope 10.

The support member 108 can define a surface 110 (e.g., 110a, 110b). In some embodiments, the surface 110 can support the mirror member 116 and/or the elevator member 124. In some examples, the surface 110 can be planar. In other examples, the surface 110 can be semi-cylindrical in shape. In some aspects, when the endoscope converter 100 is removably coupled to the endoscope 10, the surface 110 can be generally parallel to the longitudinal axis LA of the endoscope 10. In some aspects, when the endoscope converter 100 is removably coupled to the endoscope 10, the surface 110 can be generally perpendicular to the distal end 14 (e.g., planar surface of the distal end 14) of the endoscope 10.

In some embodiments, the support member 108 can include one or more channels 112 (e.g., 112a, 112b), which each form a recess into the surface 110 as illustrated in the front view of FIG. 1B. Each channel 112 can partially encompass (e.g., house) the mirror member 116 and/or the elevator member 124. In some aspects, as illustrated in FIG. 1B, a first channel 112a partially encompasses the mirror member 116 when the mirror member 116 is in a lowered position. In some examples, the depth of the first channel 112a can correspond to the thickness of the mirror member 116 such that the surface 122 of the mirror member 116 is coplanar with the surface 110 (e.g., 110a) of the support member 108 (e.g., 108a) when the mirror member 116 is in a lowered position. In some aspects, as illustrated in FIG. 1B, a second channel 112b partially encompasses the elevator member 124 when the elevator member 124 is in a lowered position. In some examples, the depth of the second channel 112b can correspond to the thickness of the elevator member 124 such that the surface 130 of the elevator member 124 is coplanar with the surface 110 (e.g., 110b) of the support member 108 (e.g., 108b) when the elevator member 124 is in a lowered position.

In some embodiments, the support member 108 can include a passageway 114, which can inhibit fluid (e.g., water, mucosal secretions) from building up in the endoscope converter 100. For example, the passageway 114 can promote fluid drainage (e.g., through the passageway 114) away from the endoscope converter 100. In some aspects, the passageway 114 can extend through the thickness (e.g., from the surface 110 to a bottom surface) of the support member 108. In some aspects, the passageway 114 can extend for a portion of the length (e.g., along the longitudinal axis LAS) of the support member 108. In some aspects, the passageway 114 can extend along the entire length of the support member 108. In some embodiments, the passageway 114 defines a right support member 108a and a left support member 108b (e.g., a clefted support member 108).

The disclosure turns now to the mirror member 116, which can transition between a lowered position (e.g., to provide a forward-facing view for the endoscope 10) and a raised position (e.g., to provide a side-facing view for the endoscope 10). The mirror member 116 extends from a first end 118 to a second end 120 and defines a longitudinal axis LAM, as illustrated for example in FIGS. 1C-1D. The mirror member 116 (e.g., first end 118) is operably coupled to the support member 108 such that the mirror member 116 can rotate with respect to the support member 108 (e.g., rotate about the first end 118).

In some aspects, the mirror member 116 can rotate (e.g., transition) between a lowered position (e.g., a horizontal position as illustrated for example in FIG. 1D) and a raised position (e.g., an elevated or angled position as illustrated for example in FIG. 1E). In the lowered position, the mirror member 116 provides forward-viewing (e.g., unobstructed viewing) through the endoscope 10 (e.g., through the objective lens 20, which can be associated with a camera). In the raised position, the mirror member 116 can provide side-viewing (e.g., reflected view reflected by the mirror member 116). In some examples, various positions (e.g., angles of rotation) of the mirror member 116 that provide forward-viewing through the endoscope 10 can be referred to as the lowered position. Similarly, in some examples, various positions (e.g., angles of rotation) of the mirror member 116 that provide side-viewing through the endoscope 10 can be referred to as the raised position.

The longitudinal axis LAM of the mirror member 116 can define an angle of rotation (e.g., angle of rotation of the mirror member 116). In some examples, the angle of rotation can be defined as the angle between the longitudinal axis LAM of the mirror member 116 and the longitudinal axis LAS of the support member 108. In some examples, the angle of rotation can be defined as the angle between the longitudinal axis LAM of the mirror member 116 and the longitudinal axis LA of the distal end 14 of the endoscope 10, which are illustrated for example in FIG. 1E. As previously discussed, in some instances the longitudinal axis LAS of the support member 108 can be generally parallel to the longitudinal axis LA of the distal end 14 of the endoscope 10.

In some embodiments, the angle of rotation of the mirror member 116 can range between approximately 0-degrees and approximately 90-degrees. In some embodiments, the angle of rotation can range between approximately 0-degrees and approximately 60-degrees. As non-limiting examples, the angle of rotation can be approximately 0-degrees, 2.5-degrees, 5-degrees, 7.5-degrees, 10-degrees, 12.5-degrees, 15-degrees, 17.5-degrees, 20-degrees, 22.5-degrees, 25-degrees, 27.5-degrees, 30-degrees, 32.5-degrees, 35-degrees, 37.5-degrees, 40-degrees, 42.5-degrees, 45-degrees, 47.5-degrees, 50-degrees, 52.5-degrees, 55-degrees, 57.5-degrees, or 60-degrees. In some examples, the angle of rotation of the mirror member 116 can be up to 52.5-degrees. In some embodiments, the angle of rotation can range between approximately 0-degrees and approximately 45-degrees.

The mirror member 116 can define an outer surface 122. In some examples, the outer surface 122 is a planar surface. The outer surface 122 can include, in whole or in part, a mirror (e.g., a mirrored surface). When the mirror member 116 is in a raised position, the mirror can reflect at least a portion of the view through the endoscope 10 (e.g., the objective lens 20), such that the mirror member 116 provides side viewing (e.g., a reflected view) through the endoscope 10. In this manner, the typically forward-viewing endoscope 10 can provide, in whole or in part, a side view.

In some embodiments, as illustrated for example in FIG. 1B, when the endoscope converter 100 is removably coupled to the endoscope 10, the mirror member 116 is substantially aligned with an objective lens 20 (e.g., substantially aligned with the camera associated with the objective lens 20) of the endoscope 10. In some aspects, the longitudinal axis LAM of the mirror member 116 can be coplanar with a longitudinal axis of the objective lens 20 (e.g., coplanar with a longitudinal axis of the camera). In some examples, the longitudinal axis LAM of the mirror member 116 is coplanar with the longitudinal axis of the objective lens 20 when the mirror member 116 is in a lowered position, when the mirror member 116 is transitioning between the lowered position and a raised position, and/or when the mirror member 116 is in the raised position. In some instances, the plane defined by the longitudinal axis LAM of the mirror member 116 and the longitudinal axis of the objective lens 20 is generally perpendicular to the surface 110 (e.g., 110a) of the support member 108.

The disclosure turns now to the elevator member 124, which can transition between a lowered position (e.g., position an instrument at no angle with respect to the to the longitudinal axis LA of the endoscope 10 at the distal end 14) and a raised position (e.g., position an instrument at an angle with respect to the longitudinal axis LA of the endoscope 10 at the distal end 14). The elevator member 124 extends from a first end 126 to a second end 128 and defines a longitudinal axis LAE, as illustrated for example in FIG. 1C. The elevator member 124 (e.g., first end 126) is operably coupled to the support member 108 such that the elevator member 124 can rotate with respect to the support member 108 (e.g., rotate about the first end 126).

In some aspects, the elevator member 124 can rotate (e.g., transition) between a lowered position (e.g., horizontal) and a raised position (e.g., elevated position). It should be noted that the elevator member 124 is not illustrated in the side views of FIGS. 1D-1E, but the elevator member 124 can rotate between a lowered position or a raised position similar to the mirror member 116. In the lowered position, the elevator member 124 can provide for instruments (e.g., tools) to pass through the distal end 14 of the endoscope 10 (e.g., the instrument channel outlet 24) generally parallel to the longitudinal axis LA of the endoscope 10. In the raised position, the elevator member 124 can provide for instruments to pass through the distal end 14 of the endoscope 10 at an angle (e.g., angle of elevation) with respect to the longitudinal axis LA of the endoscope 10.

The longitudinal axis LAE of the elevator member 124 can define an angle of elevation (e.g., angle of elevation of the elevator member 124). In some examples, the angle of elevation can be defined as the angle between the longitudinal axis LAE of the of the elevator member 124 and the longitudinal axis LAS of the support member 108. In some examples, the angle of elevation can be defined as the angle between the longitudinal axis LAE of the of the elevator member 124 and the longitudinal axis LA of the distal end 14 of the endoscope 10. As previously discussed, in some instances the longitudinal axis LAS of the support member 108 can be generally parallel to the longitudinal axis LA of the distal end 14 of the endoscope 10.

In some embodiments, the angle of elevation of the elevator member 124 can range between approximately 0-degrees and approximately 90-degrees. As non-limiting examples, the angle of elevation can be approximately 0-degrees, 2.5-degrees, 5-degrees, 7.5-degrees, 10-degrees, 12.5-degrees, 15-degrees, 17.5-degrees, 20-degrees, 22.5-degrees, 25-degrees, 27.5-degrees, 30-degrees, 32.5-degrees, 35-degrees, 37.5-degrees, 40-degrees, 42.5-degrees, 45-degrees, 47.5-degrees, 50-degrees, 52.5-degrees, 55-degrees, 57.5-degrees, 60-degrees, 62.5-degrees, 65-degrees, 67.5-degrees, 70-degrees, 72.5-degrees, 75-degrees, 77.5-degrees, 80-degrees, 82.5-degrees, 85-degrees, 87.5-degrees, or 90-degrees. In some examples, the elevation of the elevator member 124 can be up to 90-degrees.

The elevator member 124 can define an outer surface 130. In some examples, the outer surface 130 is a planar surface. The outer surface 130 can position an instrument (e.g., instrument passing through the instrument channel outlet 24 of the endoscope 10). For example, the outer surface 130 can directly contact a portion of an instrument to position it at an angle of elevation. In this manner, the elevator member 124 can provide for a side approach of instruments, wherein typical endoscopes 10 allow for only the forward approach of instruments.

In some embodiments, when the endoscope converter 100 is removably coupled to the endoscope 10, the elevator member 124 is substantially aligned with an instrument channel outlet 24 (e.g., substantially aligned with the instrument channel at the distal end 14) of the endoscope 10. In some aspects, the longitudinal axis LAE of the elevator member 124 can be coplanar with a longitudinal axis of the instrument channel outlet 24. In some examples, the longitudinal axis LAE of the elevator member 124 is coplanar with the longitudinal axis of the instrument channel outlet 24 when the elevator member 124 is in a lowered position, when the elevator member 124 is transitioning between the lowered position and a raised position, and/or when the elevator member 124 is in the raised position.

The disclosure turns now to the tension member 132 (e.g., 132a, 132b), as illustrated for example in FIG. 1A, which can be actuated to control the position (e.g., rotation) of the mirror member 116 and/or position (e.g., rotation) of the elevator member 124. In some embodiments, the angle of rotation of the mirror member 116 and the angle of elevation of the elevator member 124 can be independently controlled (e.g., a tension member 132a in communication with the mirror member 116 and a tension member 132b in communication with the elevator member 124). In some embodiments, the tension member 132 can include one tension member 132 that is in communication with both the mirror member 116 and the elevator member 124. The tension member 132 can be actuated to control both the angle of rotation of the mirror member 116 and the angle of elevation of the elevator member 124. In other embodiments, more than one tension members 132, such as two tension members 132a, 132b, can be included in the endoscope converter 100. For example, a first tension member 132a can be in communication with the mirror member 116 and can be actuated to cause the mirror member 116 to rotate, thereby adjusting or otherwise changing the angle of rotation of the mirror member 116. A second tension member 132b can be in communication with the elevator member 124 and can be actuated to cause the elevator member 124 to rotate, thereby adjusting or otherwise changing the angle of elevation of the elevator member 124. The angle of rotation of the mirror member 116 and the angle of elevation of the elevator member 124 can be independently controlled by the first tension member 132a and the second tension member 132b, respectively.

Each tension member 132 extends from a proximal end 134 (e.g., 134a, 134b) to a distal end 136 (e.g., 136a, 136b), as illustrated for example in FIG. 1A, and defines a longitudinal axis LAT, as illustrated for example in FIGS. 1D-1E. The distal end 136 of each tension member 132 can be in communication with the mirror member 116 and/or elevator member 124. When the endoscope converter 100 is removably coupled to the endoscope 10, the proximal end 134 of each tension member 132 can be located along the length of the endoscope 10. Each tension member 132 can extend along a portion of the length of the endoscope 10. In some aspects, each tension member 132 defines a length between the proximal end 134 and the distal end 136. In some aspects, the length the tension member 132 can be equal to or greater than the length of the insertion tube 12 of the endoscope 10, as illustrated for example in FIG. 1A. In some aspects, the length of the tension member 132 can be equal to or less than the distance between the distal end 14 of the endoscope 10 and the angulation controls of the endoscope 10, as illustrated for example in FIG. 1A.

The longitudinal axis LAT of each tension member 132 can be generally parallel to the longitudinal axis LA of the endoscope 10 such that the longitudinal axis LAT of each tension member 132 varies with the longitudinal axis LA of the endoscope 10 as the endoscope 10 is bent, curved, or otherwise manipulated during use. In some embodiments, as illustrated for example in FIG. 1A, each tension member 132 can extend along the outside of the endoscope 10. For example, one or more securement bands 138 can secure each tension member 132 to the endoscope 10. In some embodiments, each tension member 132 can extend along the inside of the endoscope 10 (e.g., within an instrument channel).

Each tension member 132 can be actuated (e.g., advanced or retracted) to control the angle of rotation of the mirror member 116 and/or angle of elevation of the elevator member 124. For example, actuating (e.g., advancing) the tension member 132 can cause the angle of rotation of the mirror member 116 to increase (e.g., transition from a lowered position to a raised position) and/or cause the angle of elevation of the elevator member 124 to increase (e.g., transition from a lowered position to a raised position). Similarly, actuating (e.g., retracting) the tension member 132 can cause the angle of rotation of the mirror member 116 to decrease (e.g., transition from a raised position to a lowered position) and/or cause the angle of elevation of the elevator member 124 to decrease (e.g., transition from a raised position to a lowered position).

Each tension member 132 can include a proximal control 140 (e.g., 140a, 140b), as illustrated for example in FIG. 1A, which can be manipulated (e.g., pressing a button, switching or toggling a lever, or rotating a dial) to control the actuation of each respective tension member 132 (e.g., 132a, 132b). In some examples, as illustrated in FIG. 1A, the proximal control 140 includes a lever, which can be switched or toggled to actuate the respective tension member 132. In other examples, the proximal control can include a button, dial, etc. The proximal control 140 can be in communication with the proximal end 134 (e.g., 134a, 134b) of each tension member 132. Manipulating (e.g., switching, toggling) the proximal control 140 causes the tension member 132 to actuate (e.g., advance or retract), which causes the mirror member 116 and/or elevator member 124 to rotate. Each proximal control 140 can be positioned on the outside of the endoscope 10. For example, one or more securement bands 138 can secure each proximal control 140 to the endoscope 10.

In some embodiments, one proximal control 140 is in communication with the proximal end 134 of the tension member 132 (e.g., manipulating the proximal control 140 actuates the tension member 132, which causes the mirror member 116 and the elevator member 124 to rotate). In other examples, a first proximal control 140a is in communication with the proximal end 134a of a first tension member 132a (e.g., manipulating the proximal control 140a actuates the tension member 132a, which causes the mirror member 116 to rotate) and a second proximal control 140b is in communication with the proximal end 134b of a second tension member 132b (e.g., manipulating the proximal control 140b actuates the tension member 132b, which causes the elevator member 124 to rotate).

The disclosure turns now to the one or more light sources 142 (e.g., 142a, 142b), as illustrated for example in FIG. 1C-1E, which can be configured to project light therefrom (e.g., to illuminate an area in the duodenum of the patient). In some embodiments, the light sources 142 can be illuminated (e.g., turned on) to illuminate, or otherwise provide light to, an area such as the side view area that is visible when the mirror member 116 is in a raised position. The light sources 142 can be, as non-limiting examples, light emitting diode (LED) lighting or fiber optic lighting. In some aspects, the light sources 142 can be a cold light (e.g., luminescence) source. In some aspects, any heat generated from the light sources 142 (e.g., LED light) can be dissipated through one or more heat pipes (e.g., tension members). In some examples, the heat pipes or tension members used to dissipate heat from the light sources can be separate (e.g., independent) from the tension members 132 (e.g., 132a, 132b) to control the angle of rotation of the mirror member 116 and/or angle of elevation of the elevator member 124. In some embodiments, the light source can include an outer cover (e.g., lens), which can enclose the light source 142 with respect to the endoscope converter 100.

The light sources 142 can define a longitudinal axis LAL, as illustrated for example in the side views in FIGS. 1D-1E. In some embodiments, when the endoscope converter 100 is removably coupled to the endoscope 10, the longitudinal axis LAL of the light sources 142 can face radially outward from the longitudinal axis of the endoscope, such that the light sources can illuminate at least a portion of the area that is visible from the side view provided when the mirror member 116 is in a raised position as previously discussed.

In some aspects, when the endoscope converter 100 is removably coupled to the endoscope 10, the longitudinal axis LAL of the light sources 142 can be generally perpendicular to the longitudinal axis LA of the endoscope 10, which are illustrated for example in FIGS. 1D-1E. In some aspects, when the endoscope converter 100 is removably coupled to the endoscope 10, the longitudinal axis LAL of the light sources 142 can be generally parallel to the distal end 14 (e.g., planar surface of the distal end 14) of the endoscope 10. In some aspects, when the endoscope converter 100 is removably coupled to the endoscope 10, the longitudinal axis of the light sources 142 can be generally perpendicular to the surface 110 of the support member 108 (e.g., planar surface).

When illuminated, the light sources 142 can direct lighting towards or otherwise provide lighting to a side area or portion of the side area that is visible from the side view, which can be provided when the mirror member 116 is in a raised position to provide a side view as previously discussed. When illuminated, the light sources 142 can project a beam of light that defines a longitudinal axis. The longitudinal axis of the light beam can be coincident to the longitudinal axis LAL of the light sources 142. In some examples, the endoscope converter 100 can include one light source 142. In some examples, as illustrated in FIGS. 1A-1C for example, the endoscope converter 100 can include two light sources 142 (e.g., 142a, 142b). In some examples, the endoscope converter 100 can include three or more light sources 142.

The light sources 142 can be incorporated into or otherwise coupled to the endoscope converter 100. In some embodiments, as illustrated in FIGS. 1A-1E for example, the light sources 142 can be incorporated into or coupled to the attachment portion 102 (e.g., 102a, 102b). For example, the first attachment portion 102a can house the first light source 142a and the second attachment portion 102b can house the second light source 142b. In other embodiments, which are not illustrated, the light sources 142 can be incorporated into the support member 108. In still other embodiments, which are not illustrated, the light sources 142 can be incorporated into the mirror member 116 and/or the elevator member 124.

A controller (not illustrated in the figures) in communication with the light sources 142 can be manipulated (e.g., pressing a button, switching or toggling a lever, or rotating a dial) to controller (e.g., turn on, turn off) the illumination of the light sources 142. For example, the controller can be a dial at the near the proximal end of the endoscope converter 100 (e.g., adjacent to the proximal end of the 134 of the tension member 132, adjacent to the one or more securement bands 138, adjacent to the proximal control 140). In some embodiments, the controller can adjust the intensity (e.g., more intense illumination, less intense illumination) of the light sources 142. For example, the controller can include a dimmer to adjust the intensity of the light sources 142 such that when the light sources 142 are illuminated (e.g., turned on), the intensity can be adjusted (e.g., brightened, dimmed). In some embodiments, the light sources 142 (e.g., 142a, 142b) can be independently controlled (e.g., turned on or off) and/or independently adjusted (e.g., more intense illumination, less intense illumination). In some aspects, each of the light sources 142 can have its own individual controller.

In some aspects, the light sources 142 can be illuminated (e.g., turned on) or unilluminated (e.g., turned off). For example, the light sources 142 can be unilluminated as the distal end 14 of the endoscope 10 is inserted into and advanced through the body of the patient. Then, after the endoscope 10 reaches a desired location (e.g., the duodenum) within the patient, the light sources 142 can be illuminated.

The light sources 142 can receive power from an external power source (not shown in the figures). The external power source can be, as non-limiting examples, a battery, a wall outlet (e.g., in communication with the electric grid), and/or a USB port (e.g., computer USB port on an endoscopy cart). For example, a cable 144 (e.g., power cable, USB cable) can extend from the light sources 142 controller (e.g., at the proximal end of the endoscope converter 100) to the power supply. In some instances, the power cable 144 can run from the controller associated with the light sources 142 to the proximal end of the endoscope 10 (e.g., along the endoscope 10). For example, the power cable 144 can be secured to the endoscope 10 with one or more securement bands (not shown in the figures). When the cable 144 is connected to the power supply, the cable can supply power to the light sources 142 such that the light sources can be illuminated (e.g., turned on).

FIGS. 2A-2B illustrate another exemplary embodiment of an endoscope converter 200 (e.g., endoscope viewing converter), in accordance with one or more aspects of the present disclosure. The endoscope converter 200 in FIGS. 2A-2B can include or more same or similar features as those described above with respect to the endoscope converter 100 in FIGS. 1A-1E. Due to the same or similar features, the reference numbers and corresponding description provided above for various components, elements, portions, etc., included in the endoscope converter 100 in FIGS. 1A-1E may be generally applied to the same or similar components, elements, portions, etc. included in the endoscope converter 200 described in FIGS. 2A-2B; however the reference numbers in FIGS. 2A-2B are 200 series rather than 100 series. For example, the endoscope converter 200 can be removably coupled to an endoscope 10 and used to provide alternative viewing (e.g., side viewing) from the endoscope 10.

For example, FIG. 2A is a perspective view of the endoscope converter 200, coupled to the distal end 14 of an endoscope 10, with the mirror member 216 in a lowered position and the elevator member 224 in a lowered position. In FIG. 2B, the mirror member 216 is in a raised position and the elevator member 224 is in a raised position. The mirror member 216 and/or elevator member 224 can be transitioned between the lowered position and the raised position.

The disclosure turns now to a method of using the endoscope converter 100, such as is illustrated in FIGS. 1A-1E. To use the endoscope converter 100, the endoscope converter 100 can be removably coupled (e.g., attached) to the distal end 14 of an endoscope 10 as described above. Then, the operator inserts the distal end 14 of the endoscope 10, which includes the endoscope converter 100, into the patient (e.g., the mouth of the patient). The operator can advance the endoscope 10 to a desired location (e.g., within the duodenum) within the patient. For example, the operator can advance the distal end 14 of the endoscope 10 so that it passes through the mouth of the patient, through the throat of the patient, and enters the stomach of the patient. The operator can further advance the distal end 14 of the endoscope 10 so that it passes through the stomach of the patient and enters the duodenum of the patient. For example, the endoscope converter 100 can be used with an endoscope 10 during an ERCP procedure. In one aspect of performing an ERCP procedure, the mirror member 116 and the elevator member 124 are in a lowered position both when the distal end 14 of the endoscope 10 is inserted into the mouth of the patient and when the distal end 14 of the endoscope 10 is advanced into the duodenum of the patient. After the distal end 14 of the endoscope 10 is advanced into the duodenum, the mirror member 116 and/or the elevator member 124 can be actuated (e.g., rotated) into a raised position as desired by the operator performing the ERCP procedure.

In some aspects, the operator can use various components of the endoscope 10 as desired. For example, the operator can use the objective lens 20 (e.g., and the associated camera), one or more light-guide lenses 22 (e.g., and the associated light source or light sources), the air/water nozzle 26 (e.g., and the associated air and/or water supply), the auxiliary water channel outlet 28 (e.g., and the associated auxiliary water channel), or a combination thereof to insert the distal end 14 of the endoscope 10, to advance or position the distal end 14 of the endoscope 10, and/or to retract the distal end 14 of the endoscope 10. Moreover, the operator can use the objective lens 20 (e.g., and the associated camera), one or more light-guide lenses 22 (e.g., the associated lights), the air/water nozzle 26, the auxiliary water channel outlet 28, or a combination thereof when the distal end 14 of the endoscope 10 is at the desired location (e.g., within the duodenum) within the patient.

At any time during the advancement, positioning, or otherwise placement of the endoscope 10, the operator can actuate (e.g., rotate) the mirror member 116 (e.g., by switching or toggling the proximal control 140 to actuate the tension member 132 in communication with the mirror member 116). For example, the operator can transition the mirror member 116 from a lowered position to a raised position, such that the operator can obtain at least a partial side view. Similarly, the operator can transition the mirror member 116 from the raised position to the lowered position to use the forward-viewing of the endoscope 10.

In some aspects, the mirror member 116 remains in the lowered position as the endoscope 10 is advanced into the patient, such that the operator can use the forward-viewing capabilities of the endoscope 10. In some aspects, after the distal end 14 of the endoscope 10 reaches a desired location, the mirror member 116 can be actuated (e.g., rotated) into a raised position, such that the operator can use the side-viewing capabilities of the endoscope converter 100. In some aspects, the mirror member 116 can be actuated into a lowered position before the endoscope is retracted (e.g., removed) from the patient. In some aspects, the mirror member 116 remains in the lowered position as the endoscope is retracted from the patient.

When the mirror member 116 is in a raised position, the mirror member 116 provides side viewing for the endoscope 10. This provides the operator with a side view, similar to the viewing that would be provided by a traditional side-viewing duodenoscope, when using the endoscope 10. In effect, the side facing enhances the ability of an operator to perform procedures in which side viewing is desirable or otherwise necessary (e.g., ERCP). In one embodiment, the mirror member 116 is transitioned to a raised position only temporarily, thereby temporarily converting the endoscope 10 into a side-viewing device. The operator can take advantage of both forward viewing and side viewing because the mirror member 116 can transition between a lowered position (e.g., facilitating forward viewing) and a raised position (e.g., facilitating side viewing). For example, the operator can have the mirror member 116 in a lowered position to facilitate forward viewing when the operator is advancing and/or retracting the endoscope 10. The operator can have the mirror member 116 in a raised position to facilitate side viewing when the distal end 14 of the endoscope 10 is at a desired location within the patient.

At any time during the advancement, positioning, or otherwise placement of the endoscope 10, the operator can actuate (e.g., rotate) the elevator member 124 (e.g., by switching or toggling the proximal control 140 to actuate the tension member 132 in communication with the elevator member 124). For example, the operator can transition the elevator member 124 from a lowered position to a raised position, such that the operator can advance a tool away from the endoscope 10 (e.g., through the instrument channel outlet 24) at an angle of elevation. After transitioning the elevator member 124 to a raised position, the operator can transition the elevator member 124 from the raised position to the lowered position.

In some aspects, the elevator member 124 remains in a lowered position as the endoscope 10 is advanced. In some aspects, after the distal end 14 of the endoscope 10 reaches a desired location, the elevator member 124 can be actuated (e.g., rotated) into a raised position, such that surface 130 of the elevator member 124 can position a tool. In some aspects, the elevator member 124 can be actuated into a lowered position before the endoscope is retracted (e.g., removed) from the patient. In some aspects, the elevator member 124 remains in the lowered position as the endoscope is retracted from the patient.

When the elevator member 124 is in a raised position, the elevator member 124 provides an angle of elevation. This allows tools (e.g., inserted through the instrument channel outlet 24) to be positioned at an angle of elevation (e.g., an angle relative to the longitudinal axis LA of the distal end 14 of the endoscope 10). In effect, the side facing enhances the ability of an operator to perform procedures in which side viewing is desirable or otherwise necessary (e.g., ERCP). In one embodiment, the elevator member 124 is transitioned into a raised position only temporarily, thereby temporarily converting the endoscope into a side use device.

At any time during advancement, positioning, or otherwise placement of the endoscope 10, the operator can illuminate (e.g., turn on) the light sources 142 (e.g., by rotating the proximal dial to illuminate the light sources 142). In some aspects, the lights sources 142 be unilluminated (e.g., turned off) as the endoscope 10 is advanced into the patient. In some aspects, after the distal end 14 of the endoscope 10 reaches a desired location, the light sources 142 can be illuminated. In some aspects, the light sources 142 can be unilluminated (e.g., turned off) before the endoscope 10 is retracted (e.g., removed) from the patient.

In some aspects, corresponding to the rotation of the mirror member 116 to provide a side view, the operator can illuminate the light sources 142 to illuminate, or otherwise provide light to, the area or a portion of the area visible in the side view (e.g., illuminate a portion of the duodenum). For example, when the mirror member 116 is in a raised position as illustrated for example in FIG. 1E, the lights sources 142 can be illuminated. In this manner, with the mirror member 116 in a raised position and the light sources 142 illuminated, the operator is provided with a side view that includes lighting directed to the area of viewing.

What is claimed is:

1. An endoscope converter for use with an endoscope, the endoscope converter comprising:
a support member;
an attachment portion operable to removably couple the support member to the endoscope; and
a mirror member operable to rotate with respect to the support member having a first end, a second end, and an outer surface, wherein the first end of the mirror member is operably coupled to the support member such that the mirror member is operable to rotate about the first end of the mirror member, wherein the outer surface of the mirror member includes a mirror.

2. The endoscope converter of claim 1, wherein the mirror member is aligned with an optical lens of a camera of the endoscope when the endoscope converter is removably coupled to a distal end of the endoscope.

3. The endoscope converter of claim 2, wherein a longitudinal axis of the mirror member is coplanar with a longitudinal axis of the optical lens of the camera.

4. The endoscope converter of claim 1, wherein the mirror member is operable to rotate between a lowered position and a raised position.

5. The endoscope converter of claim 4, wherein the mirror member allows a forward view through an optical lens of a camera of the endoscope when the mirror member is in the lowered position.

6. The endoscope converter of claim 5, wherein the mirror member reflects at least a portion of the forward view to provide a side view when the mirror member is in the raised position.

7. The endoscope converter of claim 1, wherein an angle of rotation is defined between a longitudinal axis of the mirror member and a longitudinal axis of the support member, wherein rotating the mirror member causes a change in the angle of rotation of the mirror member, wherein the angle of rotation is up to 52.5-degrees.

8. The endoscope converter of claim 1 further comprising:
an elevator member having a first end, a second end, and an outer surface, wherein the first end of the elevator member is operably coupled to the support member such that the elevator member is operable to rotate about the first end of the elevator member, wherein the outer surface of the elevator member is operable to position an instrument.

9. The endoscope converter of claim 8, wherein the elevator member is aligned with an instrument channel of the endoscope when the endoscope converter is removably coupled to a distal end of the endoscope, wherein a longitudinal axis of the elevator member is coplanar with a longitudinal axis of the instrument channel.

10. The endoscope converter of claim 8, wherein an angle of elevation is defined between a longitudinal axis of the elevator member and a longitudinal axis of the support member, wherein rotating the elevator member causes a change in the angle of elevation of the elevator member.

11. The endoscope converter of claim 8 further comprising:
a first tension member having a proximal end and a distal end, the distal end of the first tension member in communication with the mirror member, wherein actuating the first tension member causes the mirror member to rotate; and
a second tension member having a proximal end and a distal end, the distal end of the second tension member in communication with the elevator member, wherein actuating the second tension member causes the elevator member to rotate.

12. The endoscope converter of claim 11 further comprising:
a first lever at the proximal end of the first tension member, wherein manipulating the first lever causes the first tension member to actuate; and
a second lever at the proximal end of the second tension member, wherein manipulating the second lever causes the second tension member to actuate.

13. The endoscope converter of claim 1, wherein the attachment portion comprises a first semi-cylindrical surface and a second semi-cylindrical surface, wherein the first semi-cylindrical surface and the second semi-cylindrical surface are positioned on opposite sides of an outer cylindrical surface at a distal end of the endoscope.

14. The endoscope converter of claim 1 further comprising:
a light source having a longitudinal axis and configured to illuminate an area aligned with the longitudinal axis of the light source, wherein the longitudinal axis of the light source is generally perpendicular to a longitudinal axis of a distal end of the endoscope when the attachment portion is removably coupled to the endoscope and the light source is illuminated.

15. The endoscope converter of claim 1, wherein the support member defines a planar surface, wherein the planar surface of the support member is generally parallel to a longitudinal axis of a distal end of the endoscope when the endoscope converter is removably coupled to a distal end of the endoscope.

16. The endoscope converter of claim 15, wherein the support member extends laterally outward from the distal end of the endoscope when the endoscope converter is removably coupled to the distal end of the endoscope, wherein the support member includes a passageway such that the support member is cleft.

17. A method of using an endoscope converter to provide side viewing with an endoscope, the method comprising:
attaching the endoscope converter to a distal end of the endoscope, the endoscope converter comprising an attachment portion, a support member, and a mirror member, wherein attachment portion is configured to attach the endoscope converter to the distal end of the endoscope, wherein the support member extends outward from the attachment portion, wherein a first end of the mirror member is rotatably coupled to the support member such that the mirror member can rotate with respect to the support member;
inserting the distal end of the endoscope into a patient, the endoscope including an optical lens at the distal end;
advancing the endoscope to a desired location within the patient;
rotating the mirror member; and
removing the endoscope from the patient.

18. The method of claim 17, wherein the mirror member is in a lowered position to allow a forward view through the optical lens of the endoscope when advancing the endoscope to the desired location.

19. The method of claim 18, wherein the mirror member is rotated into a raised position to at least partially reflect the forward view through the optical lens to provide a side view after advancing the endoscope to the desired location.

20. The method of claim 17 further comprising:
actuating an elevator member, wherein the elevator member has a first end, a second end, and an outer surface, wherein the first end of the elevator member is operably coupled to the support member such that the elevator member is operable to rotate about the first end of the elevator member, wherein the outer surface of the elevator member is operable to position an instrument.

21. The method of claim 17 further comprising:
inserting the distal end of the endoscope into a mouth of the patient; and
advancing the distal end of the endoscope through the mouth of the patient, through a throat of the patient, through a stomach of the patient, and into a duodenum of the patient.
